(19) United States
(12) Patent Application Publication
(10) Pub. No.: US 2008/0249552 A1
(43) Pub. Date: Oct. 9, 2008

(54) Working tool for medical purposes with rotating blade of adjustable size and a method thereof

(76) Inventors: Eliahu Eliachar, Haifa (IL); Nir Lilach, Kfar Yehoshua (IL); Ofer Yossepowitch, Petach Tikva (IL); Dani Sade, Kibutzuet-Alpha (IL); Eyal Bressler, East Binyamin (IL)

Correspondence Address: SCHWEITZER CORNMAN GROSS & BONDELL LLP, 292 MADISON AVENUE - 19th FLOOR, NEW YORK, NY, 10017, US

(21) Appl. No.: 11/508,757

(22) Filed: Aug. 23, 2006

Related U.S. Application Data
(63) Continuation of application No. PCT/IL05/00226, filed on Feb. 23, 2005.

(30) Foreign Application Priority Data
Feb. 23, 2004 (IL) 160517

(51) Int. Cl. A61B 17/32

(52) U.S. Cl. 606/171; 606/167; 606/170

(57) ABSTRACT

The present invention relates to a cost effective working tool useful for lateral resection of biological tissues by means of a flexible blade with an adjustable curve size. Said rod-like tool has a distal end, adapted to be inserted into a body cavity, and a proximal end, located adjacent to a user. The longitudinal axis of said maneuverable blade is parallel to the longitudinal axis of the said tool or shifted in respect to the same. The working tool comprising a bending mechanism, adapted to adjust the bending of said blade to a predetermined measure and a rotating mechanism, adapted to rotate said blade in a lateral, bi-directional movement in respect to the resected tissue such that a side-to-side resection is obtained. A novel method of lateral resecting of biological tissues by the same is also presented.

This application is a continuation of PCT/IL2005/000226 filed Feb. 23, 2005.

### FIELD OF THE INVENTION

The present invention generally relates to a working tool useful for medical purposes with a rotating blade of adjustable size. More specifically, the present invention relates to either a mechanical or diathermical reusable and/or disposable working tool adapted for flexible and/or rigid endoscopes. Said tool is used for side-to-side resection and biopsy of biological tissues from within body cavities.

### BACKGROUND OF THE INVENTION

Diagnostic and therapeutic endosocopy is commonly used to gain access to body cavities, such as the gastrointestinal tract, the lungs and the urinary tract, for the purposes of observing or removing tissues. Commercially available endoscopes are utilized either via laparoscopic procedures inserted via hollow facilitating means, e.g. trocars, laparoscopic tunnels, etc., through a body orifice such as the mouth, anal orifice, or urological orifice. Endoscopic resection has gained more and more importance in the treatment of early stage cancers over the past few years. The choice between the different available techniques depends on the site, the macroscopic type of the lesion and the personal experience of the endoscopic surgeon. Endoscopic methods for obtaining tissue samples include the use of snares, forceps, needles, brushes and scissors. While these techniques permit the accomplishment of many diagnostic and/or therapeutic goals, in some instances they may be inadequate.

For example, endoscopic removal of flat lesions may not be possible due to lack of satisfactory tools that permit complete tissue removal and enable accurate pathological assessment. For the purpose of endoscopic mucosal resection (EMR), different techniques have recently been developed, including the “lift and cut” resection, polypectomy after a submucal injection of saline, glucose and the “pure cut and hemoclip” method.

Furthermore, snares are commonly used to remove a polypoid type lesion from within the gastrointestinal tract. A common disadvantage of this technique relates to the need to maneuver the snare about the polyp, which may become a difficult and challenging task at certain locations along the gastrointestinal lumen. Commercially available rotating snares are still hampered by their jerky-type rotational movement, rendering their use mostly inconvenient for the practicing endoscopic surgeon.

Surgical knives and diathermia wires with a single maneuver, either a rotating movement or a deflecting movement are known in the art. Hence, U.S. Pat. No. 6,540,695 to Burbank et al. discloses a biopsy device characterized by a single rotating maneuver. This device has a side-cutting mechanism, which includes both a cutting wire, configured to be rotated about the longitudinal axis of a shaft, thereby isolating a body of target tissue, and a tissue anchoring mechanism. Similarly, U.S. Pat. No. 5,415,656 to Tihon et al. provides an RF electric current driven apparatus, useful for incision of a stricture within the lumen, essentially including (a) an electrically conducting, deflectable wire, combined with an introducer means for introducing the wire into the body lumen, (b) means for deflecting the proximal portion of the wire outwards relative to the introducer means, the wire being slidable within said conduit, the portion of wire deflected outwards is in the form of a loop defining a monopolar electrosurgical knife.

Such instruments are clumsy to handle and not suitable for the aforementioned delicate medical procedures. It is thus the object of this invention to provide an improved method for the removal of tissues within a body cavity. A cost effective surgical device, and especially a disposable one, adapted for lateral resection of biological tissues in or adjacent to narrow body cavities and lumens by means of a flexible blade or wire with an adjustable curve size thus remains a long felt need.

### SUMMARY OF THE INVENTION

It is thus the core of the present invention to provide a novel working tool for lateral resection of biological tissues by means of a flexible blade with adjustable curve size. Said rod-like tool has a distal end, adapted to be inserted into a body cavity, and a proximal end, located adjacent to a user; the longitudinal axis of said maneuverable blade is parallel to the longitudinal axis of the said tool or shifted in respect to the same; said working tool comprising: bending mechanism, adapted to adjust the bending of said blade to a predetermined measure; and rotating mechanism, adapted to rotate said blade in a lateral bi-directional movement in respect to the resected tissue, such that a side-to-side resection is obtained. Said working tool is especially useful for endoscopic resections.

It is in the scope of the present invention to provide the working tool as defined above and described in any of the appended figures. This working tool is especially adapted for either cold resection; wherein the maneuverable blade is a sharp razor-like member, which is adapted to cut biological tissues mechanically; or for facilitated (hot) resection by diathermia; cryo-therapy, RF or any other vibrational means; or any combination thereof; wherein the said blade is not necessarily a sharp member.

It is also in the scope of the present invention wherein the aforementioned working tool additionally comprises a plurality of maneuvering means located at its proximal portion. These means are adapted to maneuver the flexible blade, which is located at the distal end by bending it and/or rotating it laterally. Preferably yet not solely, these means are selected from at least one control knob, handle, endless cable or any combination thereof.

According to one specific embodiment of the present invention, the said working tool is additionally comprised of at least one handle located at its proximal portion. This handle is adapted to maneuver the flexible blade located at the distal end by both bending and rotating it laterally.

It is according to yet another embodiment of the present invention wherein the working tool, as defined in any of the above, comprises inter alia (a) a rectangular rigid or non-rigid rod located at the extreme distal portion of said tool; (b) a flexible blade, whose distal end is rigidly immobilized to said rod and whose proximal end comprises a rectangular hole fitted around said rectangular rod by means of a sliding bore with a polygonal profile, such that said blade can slide along its longitudinal axis solely to a predetermined point; (c) a control box comprising inter alia a housing element, and at least one control knob; (d) an endless movement cable connecting the rectangular rod at the distal portion of the tool with the control knob located at its proximal portion; and (e), an outer flexible tube with a circular bore encompassing the proximal portion of said rectangular rod and said movement cable. By pushing or pulling aforesaid control knob along the longitudinal main axis of the tool, said movement cable and said rectangular rod are simultaneously pulled or pushed, curving or flattening the said flexible curve. Moreover, by twisting said control knob around said longitudinal main axis, said movement cable and said rectangular rod are simultaneously rotated, rotating the said flexible curve on a predetermined course.

The working tool defined above may also be comprised of electrical inlet adapted to provide a blade effective for diathermia, cryo-therapy or RF means. A free and continuous electrical communication is provided in the course of the said electrical inlet to the movement cable, rectangular rod and flexible blade. This cable of the electrical inlet may be rolled over an electrical inlet cable allowing the movement cable to rotate freely. The electrical inlet may further be in communication either with a coal pressure contact and its contact spring or with a plurality of connecting plates.

It is further in the scope of the present invention wherein at least a portion of the movement cable is a spring assembly comprising a clockwise coiled inner spring and counter-clockwise coiled outer spring enveloping said second spring of the inner core and vice versa.

It is further in the scope of the present invention wherein the control box comprises inter alia (a) at least one rotating knob useful for rotating the blade in any predetermined lateral maneuver; and (b), at least one bending knob adapted to be twisted on the bending knob threads for bending it to obtain a predetermined curve. When the bending knob is twisted backwards, the movement cable or spring assembly and rectangular rode are pulled backwards, so that the flexible blade is forced to bend along its longitudinal axis to form a curved blade.

It is still in the scope of the present invention wherein the control box comprises inter alia (a) a bending knob twistable on a bending knob therefor bending it to obtain a predetermined curve; and further wherein said bending knob is twisted backwards, pulling the movement cable or spring assembly and rectangular rode backwards, so that the flexible blade is forced to bend along its longitudinal axis to form a curved blade; and a handle assembly. This handle assembly is comprised inter alia of (a) a handle maneuvered forwards and backwards along the longitudinal axis in a recess in the housing element; (b) a tube pusher with a tube-pushing thread and a protruding pin member adapted to fit a slot in the tool's housing; (c) a twisted rectangle; (d) a cable and screw connector which rigidly connects said twisted rectangle to the movement cable or spring assembly; (e) a sliding bolt; (f) at least one returning spring adapted to either pull or push said handle after its maneuver; and (g), a palm rest located at the extreme proximal end, adapted to accommodate the thumb or palm of the user. When the handle is maneuvered by the user along the longitudinal axis of the tool, it simultaneously pushes or pulls the sliding bolt, thus rotating the twisted rectangle and transforming a linear movement into a rotational maneuver of the blade, so that a side-to-side resection is provided.

The working tool as defined above is further proved useful when a blade band plate is located at the distal portion of the rod. This plate is maneuvered either by pulling or pushing of a blade bend spring by means of at least one blade bend cable comprising two proximal ends: a blade bending cable end and a blade rotating cable end. The said blade bending cable is maneuvered at its proximal end in a direction parallel to the longitudinal axis of the tool, releasing said spring and thus pushing the plate forward so that the blade is bended in a predetermined manner; and further wherein the blade rotating cable, being located at said tool's proximal portion, is pushed backwardly in a direction parallel to the longitudinal axis. Said cable, via bend cables connector and a rectangular rod connected to it, rotates a twisted rectangular rod by means of a twisted rectangular nut and a nut connector rotating assembly, such that said nut connector is in communication at its distal portion with a cap tube, and such that said cable longitudinal movement is translated by means of said blade into a predetermined lateral rotating movement.

It is a second object of the present invention to provide a method for lateral resection of biological tissues by means of the working tool as defined in any of the above, comprising a flexible blade with adjustable curve size. This method essentially includes the following steps: (a) inserting the distal end of said tool into a body cavity, and positioning the maneuverable blade in contact with or adjacent to the tissue to be resected; (b) bending said blade to a predetermined measure; (c) rotating said blade in a lateral movement such that a side-to-side resection is obtained; and (d), retracting said blade and withdrawing the tool outside said body cavity.

It is also in the scope of the present invention to provide the aforesaid method adapted for facilitated (hot) lateral resection, comprising the steps of (a) inserting the distal end of said tool into a body cavity, and positioning the maneuverable blade in contact with or adjacent to the tissue to be resected; (b) bending said blade to a predetermined measure; (c) supplying high frequency electrical current to said blade while rotating said blade in a lateral movement such that a side-to-side resection is obtained; (d) switching off the current when the incision procedure is completed; and, then (e), retracting said blade and withdrawing the tool outside said body cavity.

### BRIEF DESCRIPTION OF THE INVENTION

In order to understand the invention and to see how it may be implemented in practice, a plurality of preferred embodiments will now be described, by way of non-limiting example only, with reference to the accompanying drawings, in which

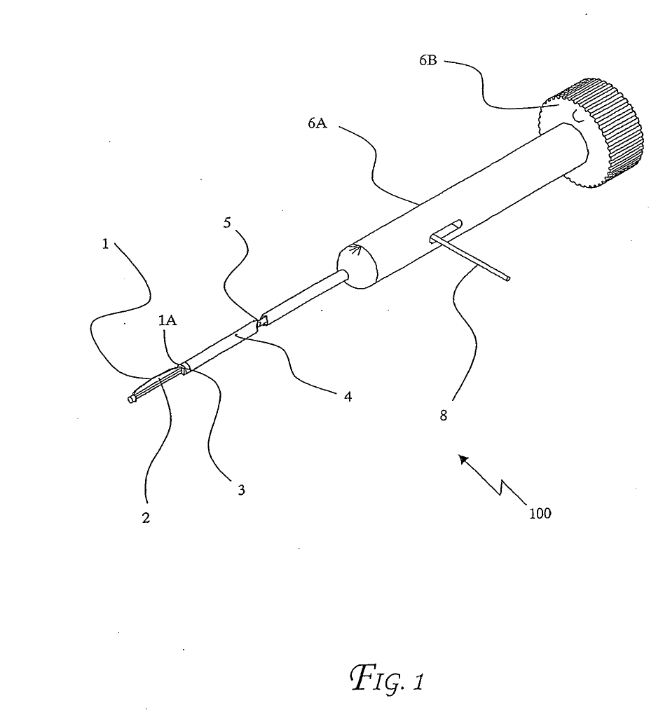

FIG. 1 schematically presents a generalized presentation of a working tool (100) according to one embodiment of the present invention in its most simplified presentation;

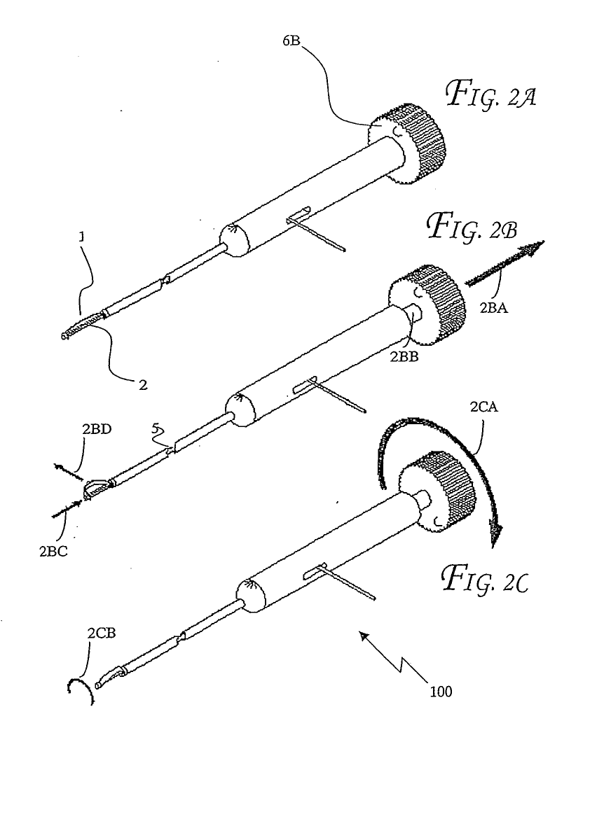

FIGS. 2A, 2B and 2C schematically present the mechanical functional relations between control knob (6B) and flexible blade (1) and rectangular rigid rod (2) and all parts of the aforementioned simplified working tool (100);

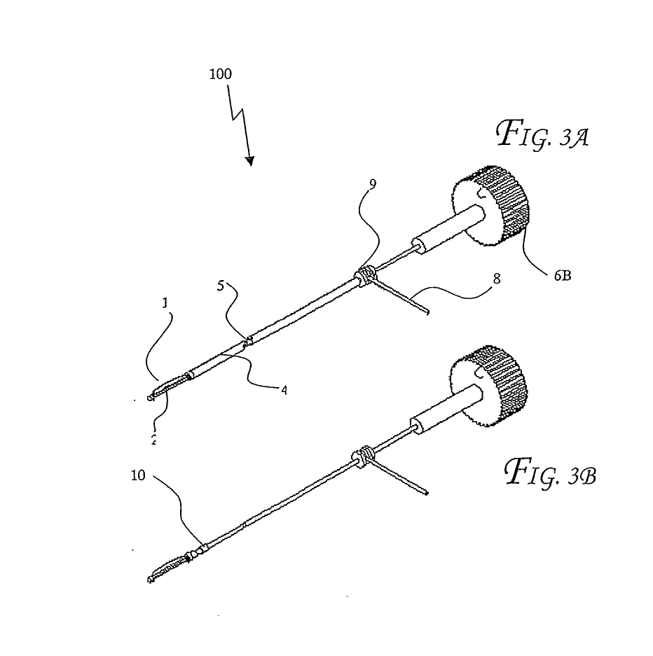

FIGS. 3A and 3B schematically present the aforementioned simplified working tool (100) without the outer housing envelope (6A);

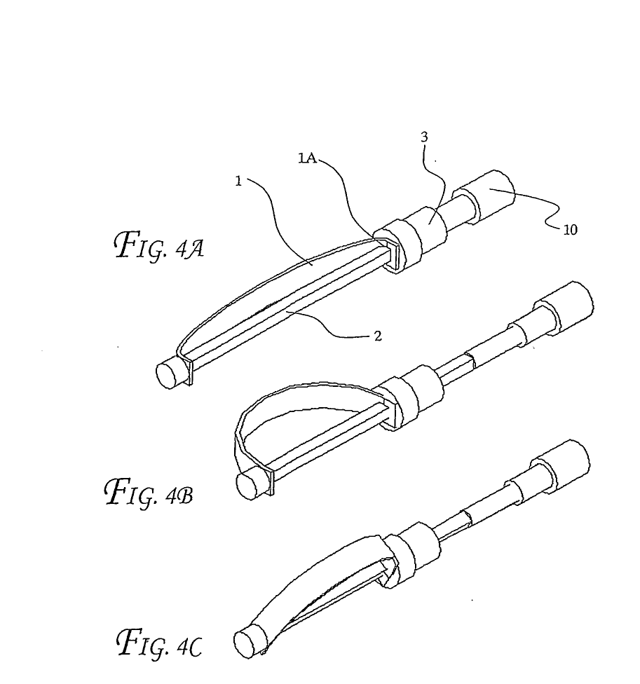

FIGS. 4A, 4B and 4C schematically illustrate the distal portion of the aforementioned working tool (100), without presenting the outer flexible tube (4).

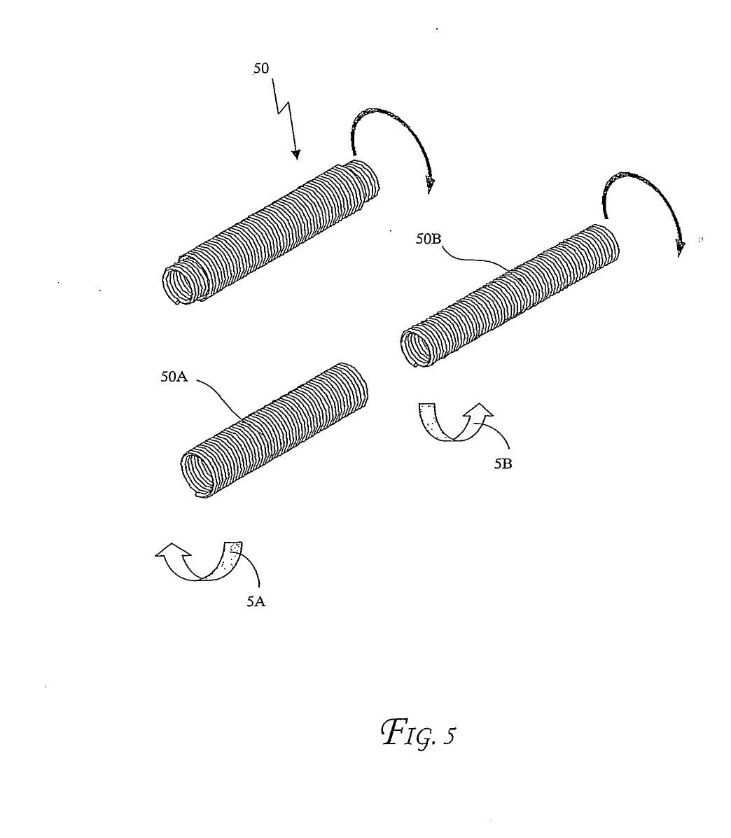

FIG. 5 schematically illustrates another embodiment of the present invention wherein the movement cable (5) is a spring assembly (50) comprising a clockwise coiled inner spring (50B) and counter-clockwise coiled outer spring (50A) enveloping said first spring;

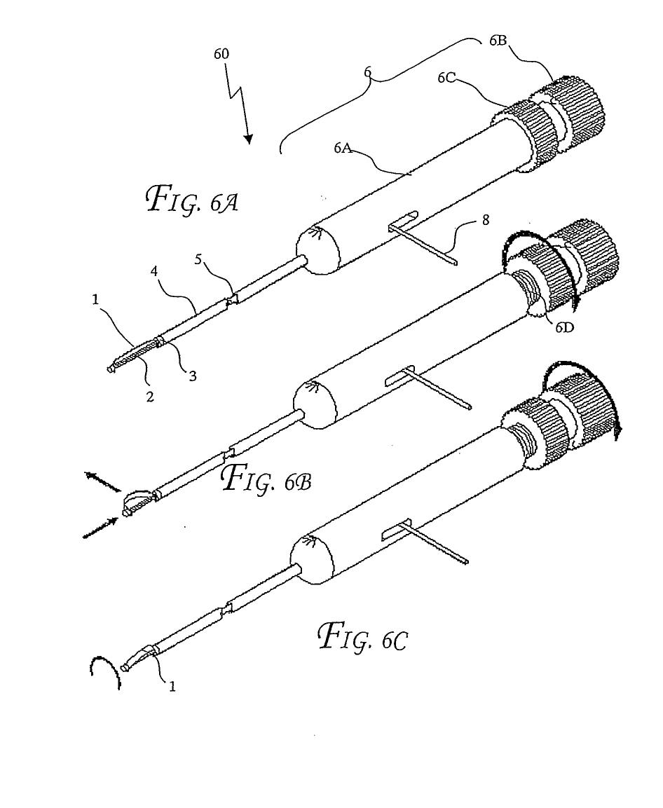

FIGS. 6A and 6B and 6C schematically illustrate the working tool (60) according to yet another embodiment of the present invention with two knobs, and the mechanical functional relations between control knobs (6B and 6C) and flexible blade (1) and rectangular rigid rod (2);

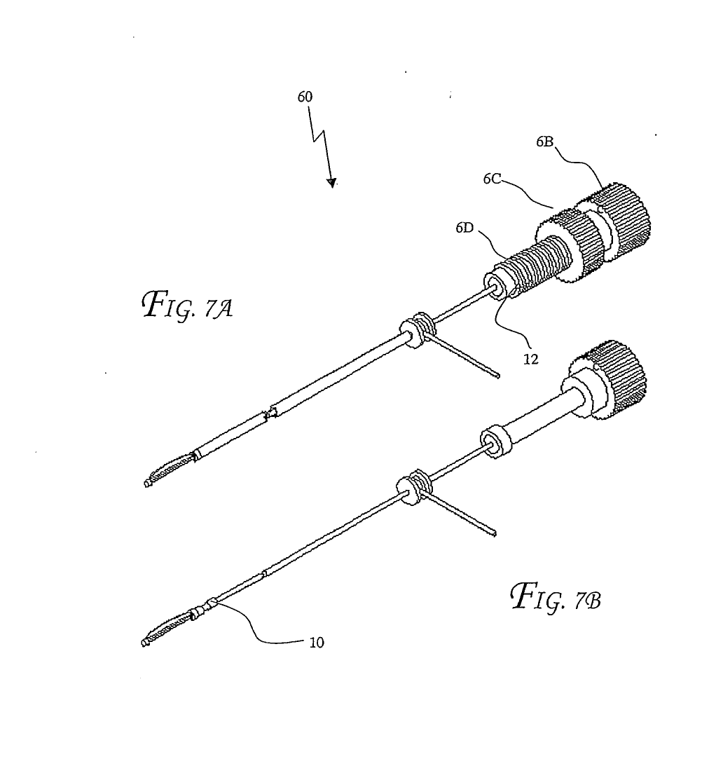

FIG. 7A, schematically presents the aforementioned working tool (60) without the box housing (6A); FIG. 7B schematically presents the aforementioned working tool (60) without the box housing (6A), bend knob (6C), and outer flexible tube (4);

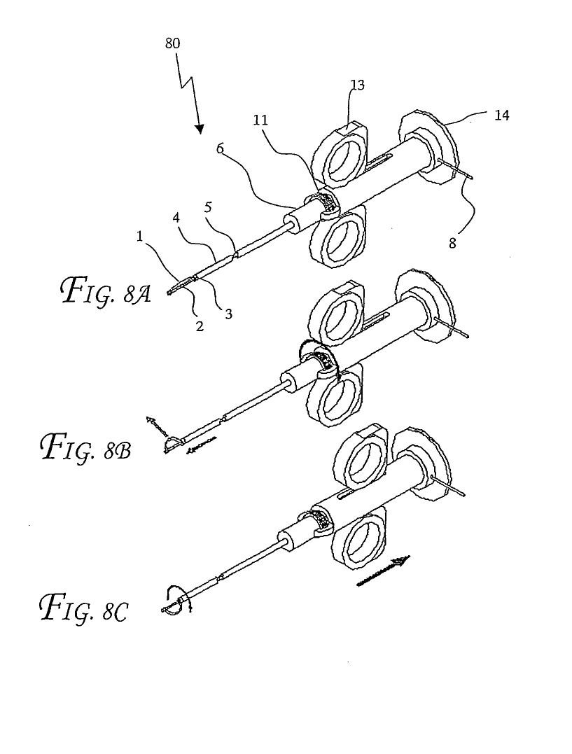

FIG. 8A, schematically presents the working tool (80) according to yet another embodiment of the present invention with a handle and a knob; FIG. 8B shows working tool (80) performing a bending maneuver; FIG. 8C illustrates the same, performing a rotating maneuver;

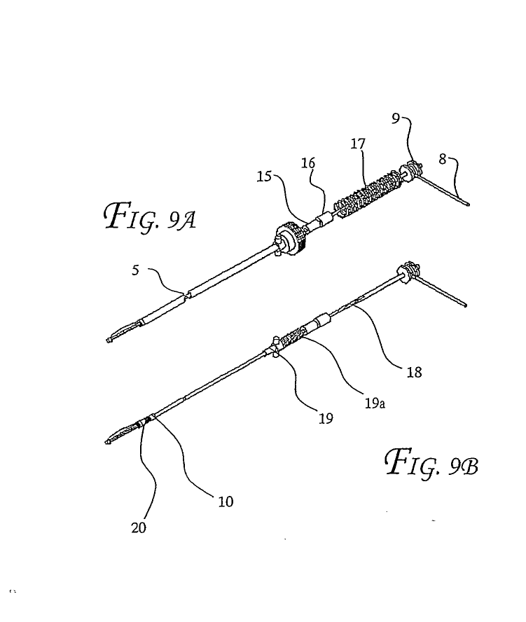

FIG. 9A, schematically presents the aforementioned working tool (80) without showing the box housing; FIG. 9B schematically presents the aforementioned working tool (80) without showing the handle, the return spring, bend knob and outer flexible tube (4);

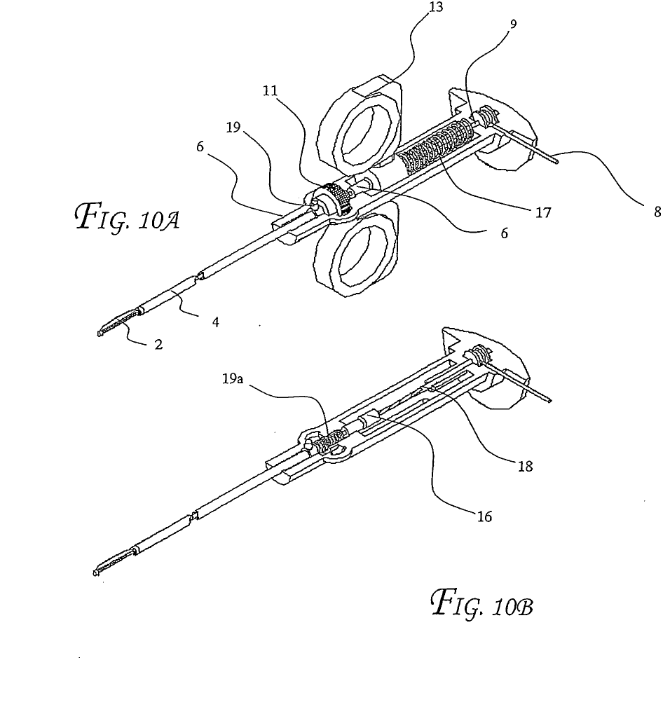

FIG. 10A schematically presents a lateral cross-section of the whole aforementioned working tool (80); FIG. 10B presents the same without the handle, return spring and bend knob;

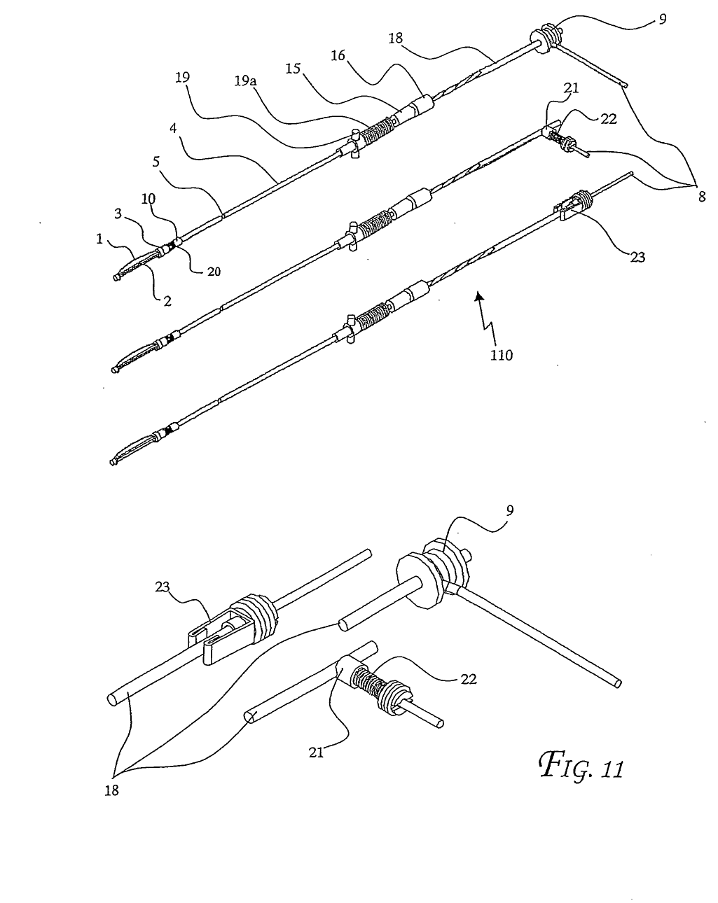

FIG. 11 schematically presents said central rotating and bending mechanism of the working tool (80) connected at its proximal portion to three different optional electrical contact assemblies; this view provides assembled and disassembled view of the same;

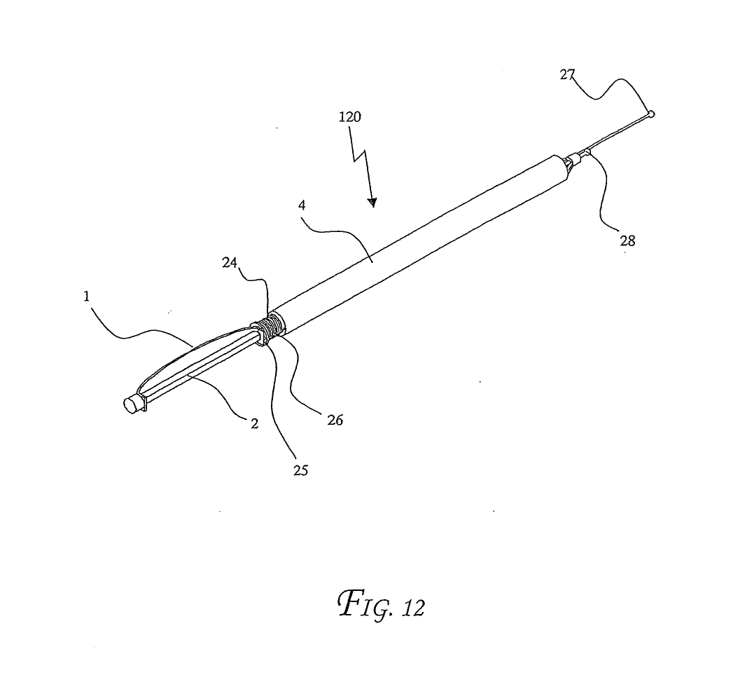

FIG. 12 schematically presents the working tool (120) according to yet another embodiment of the present invention with a blade bending cable end (27) and a blade rotating cable end (28);

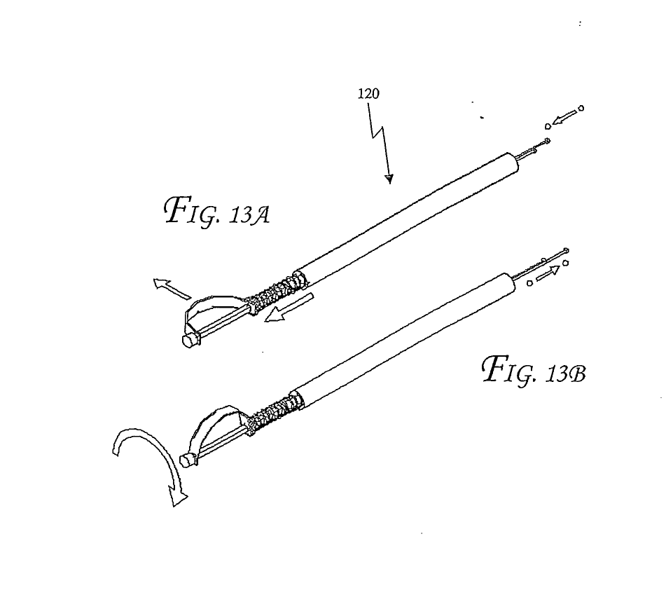

FIGS. 13A and 13B schematically present the working tool (120) mode of action;

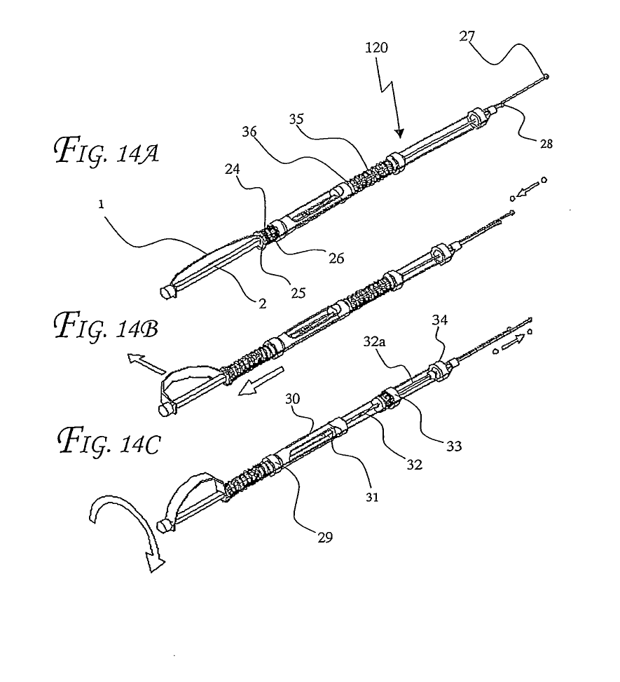

FIGS. 14A-14C schematically present said cable-based working tool (120), inner construction and mode of action;

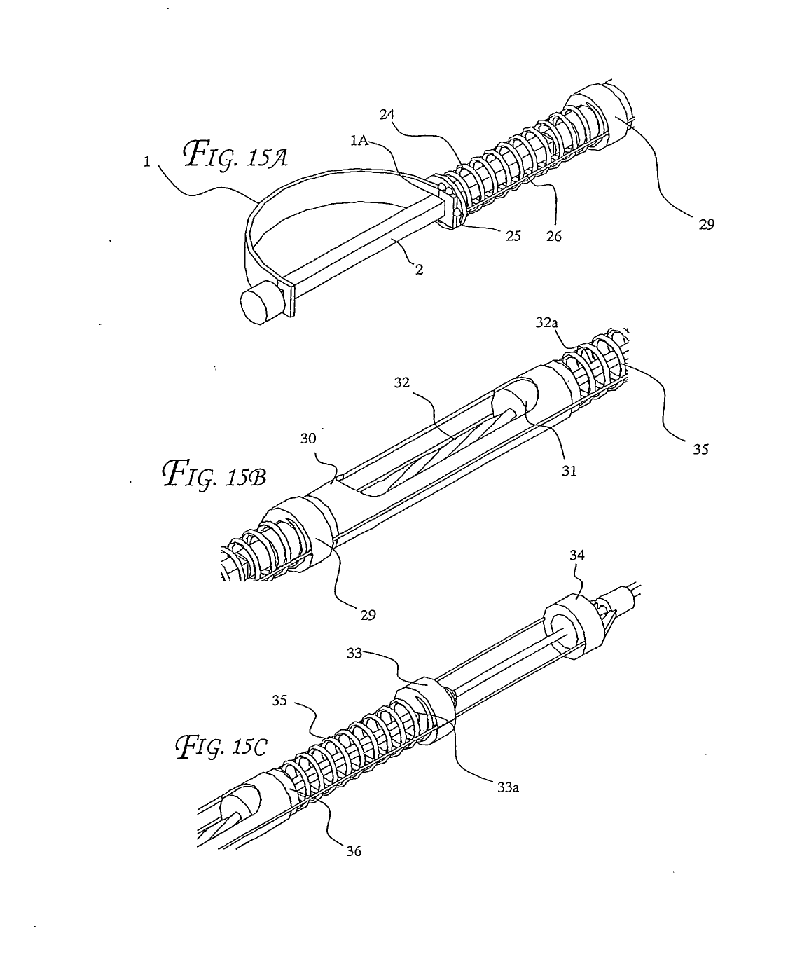

FIGS. 15A-15C schematically present a close and detailed view of the mechanisms of said cable-based working tool (120); and,

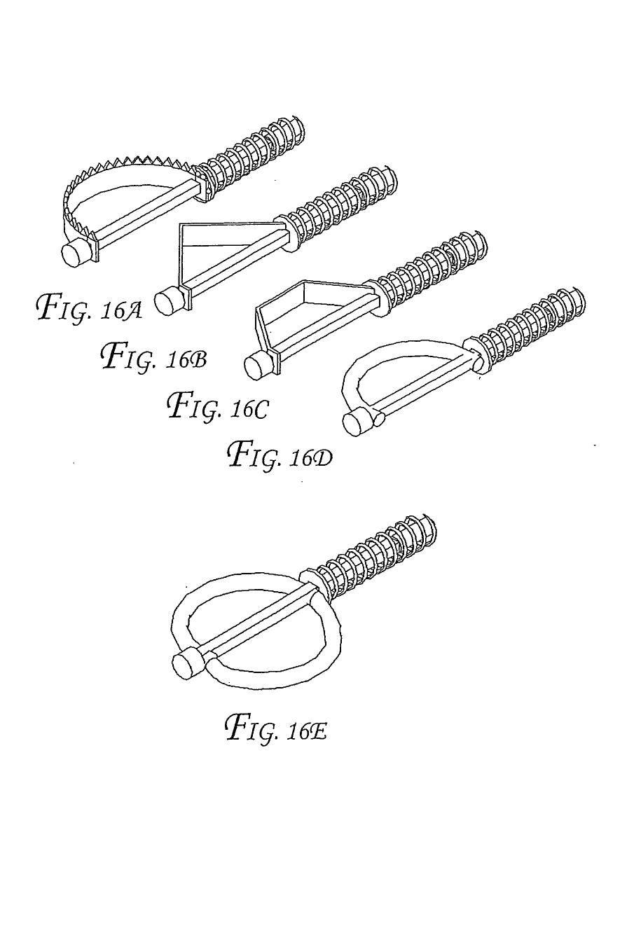

FIGS. 16A-16E schematically present examples of various blades adapted to the working tool according to various embodiments of the present invention.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a working tool for lateral resection of biological tissues by means of a flexible rotatable blade with adjustable curve size and a method of lateral resecting thereof.

The term “urological procedures” refers hereinafter to diagnostic or therapeutic procedures, performed by means of the working tool, on urological tissues in the urinary tract of male or female mammals, including the kidneys, ureters, urinary bladder, urethra, and in addition, diagnostic or therapeutic procedures, performed on the urological tissues and/or the male or female reproductive organs, including the testes, epididymis, vas deferens, seminal vesicles, prostate and penis, uterus, ovaries, Fallopian tubes, vagina and wherein such procedure is performed by inserting such working tool into the urethra or by performing a laparoscopic incision in the body for the purpose of introducing a such working tool into the urinary tract or the male or female reproductive organs.

The term “non-urological procedures” refers hereinafter any and all diagnostic or therapeutic procedures performed on mammals, by means of the working tool, on body tissues other than urological tissues, in any bodily organs, excluding the urinary tract and male or female reproductive organs, wherein such procedure is performed by inserting such working tool by any means, other than into the urethra or by performing a laparoscopic incision in the body for the purpose of introducing a such working tool into the urinary tract or the male or female reproductive organs.

The term “gastrointernal procedures” refers hereinafter to diagnostic or therapeutic procedures, performed by means of the working tool, within the gastrointestinal tract, being the alimentary canal, i.e., that musculo-membranous tube about thirty feet in length, extending from the mouth to the anus; including the upper gastrointestinal tract including the buccal cavity, the pharynx, the esophagus, the stomach, the duodenum, and the jejunum; and the lower gastrointestinal tract, including the small intestine, and the large intestine, including the cecum to the rectum, wherein the term “buccal cavity” means the mouth or oral cavity which is continuous with the integument of the lips and with the mucous lining of the pharynx; wherein the term “pharynx” relates to the part of the upper gastrointestinal tract which is placed behind the nose, mouth and larynx, being a mucomembraneous tube about 4 inches in length and posteriority with the esophagus; wherein term “esophagus” as used herein is a muscular canal about nine inches long extending from the pharynx to the stomach; wherein the term “stomach” as used herein means that part of the gastrointestinal tract between the esophagus and the small intestine, wherein such procedure is performed by inserting such working tool into the nose, mouth and/or anal body openings or by performing a laparoscopic incision in the body for the purpose of introducing a such working tool into said gastrointestinal tract.

The term “respiratory procedures” refers hereinafter to diagnostic or therapeutic procedures, performed by means of the working tool, within the respiratory system, including the nasal passageways, mouth, nostrils, nasal cavity, pharynx (naso-, oro-, laryngo-), larynx (voice box), the tonsil tissue, the lungs, trachea and bronchial passageways, and airways leading to or located in the lung, including, trachea, thoracic cavity, bronchi and alveoli wherein such procedure is performed by inserting such working tool into the nose, mouth and/or anal body openings or by performing a laparoscopic incision in the body for the purpose of introducing a such working tool into said respiratory system.

The tool is elongated and narrow and has a distal end, which is inserted into a body cavity, and a proximal end, which is adjacent to a user. It is characterized by a maneuverable blade whose longitudinal axis is parallel to the longitudinal axis of the said tool. It is comprised of a bending mechanism adapted to bend said blade to any desired curve within the designated mechanical range, and of a rotating mechanism adapted to rotate said blade in a lateral movement such that a side-to-side resection may be obtained.

Such a working tool according to the present invention refers to either a rigid or flexible tool, disposable or reusable, useful for various endoscopic devices and surgical utilizations, such as for colonoscopes, resectoscopes, cystoscopes, laparascopy, endoscopy, gastroscopy, bronchoscopy and any medical or veterinary means for pedunculated and sessile tumors removal, invasive diagnosis, biopsy and treatment, especially in the field of urology, gynecology, arthroscopy, laparoscopy endogastrology, airway management, and ENT (e.g., for otorhinolaryngology etc).

The tool comprises at least one flexible and deflectable blade, characterized by any thin, narrow and elongated shape whose longitudinal axis is parallel to the longitudinal axis of the tool. This blade is designed to bend or to curve up to a determined measure only along its said longitudinal axis.

Reference is now made now to FIG. 1, illustrating a schematic and generalized presentation of the aforementioned working tool in its most simplified presentation. Working tool (100) is adapted to be inserted into a body cavity in such a manner that a rotatable flexible blade (1) is located at the extreme distal end inside said cavity and the control box is located outside said cavity, adjunct to the user. The tool is characterized by an elongated tube-like shape with a main longitudinal axis.

The flexible blade (1) is connected to a rectangular rigid rod (2) at its extreme distal end, wherein its proximal end comprises a rectangular hole encircling the rectangular rod (2), thus enabling blade (1) to slide along the longitudinal axis of rod (2) while bending, and simultaneously preventing the blade (1) from rotating angularly around rod (2). Said rectangular rigid rod (2) extends from the extreme distal end of the tool (100) and enters flexible tube (4) through a circular hole in cup (3). Cup (3) is an adaptor located at the distal end of tube (4). Within tube (4) close to its distal end, rectangular rod (2) couples to movement cable (5) by means of connector (10, See FIG. 9B). Tube (4) connects to control box (6A). Within tube (4) and control box (6A) movement cable (5) connects rectangular rigid rod (2) at the distal portion of tool (100) with control knob (6B) at the proximal portion of tool (100). Box (6) comprises inter alia housing (6A), at least one electrical inlet (8) adapted to provide blade (1) with effective diathermia means.

It is acknowledged in this respect that flexible blade (1) is immobilized to the rectangular rode (2) at the extreme distal end or at adjacent location, wherein the proximal portion is free to slide along said rod. Nevertheless, in an alternative configuration, blade (1) is immobilized to the rod (2) a the proximal end of the rod, such that the distal portion of the blade is free to slide along the longitudinal axis up to a stopper located at the distal end of the rod. Solely for the sake of simplicity, only the first hereto-defined embodiment will be described.

Reference is made now to FIG. 2A, illustrating the aforementioned simplified working tool (100), comprising inter alia a flexible blade (1), rectangular rigid rod (2) and control knob (6B).

With reference now to FIG. 2B, a first means for maneuvering blade (1); i.e., bending it, is illustrated. Hence, rectangular rigid rod (2) may be either pulled or pushed along the longitudinal main axis (2BC). By either puffing or pushing rod (2), blade (1) bends or flattens only at its longitudinal axis. The movement cable (5) connects rod (2) to control knob (6b) through tube (4) and the operating control box (6A). When control knob (6b) is distanced from box (6a) in the direction (2BA) and to the distance (2BB), movement cable (5) is pulled accordingly, thus pulling rod (2) into tube (4) through cap (3), and accordingly the distal end of flexible blade (1) is pulled in the direction (2BA) and (2BC). Blade (1) cannot enter cap (3) and thus bends in the direction (2BD) respectively and vice versa. The aforementioned bending of blade (1) is thus provided by a controllable blade curve having longitudinal and lateral dimensions of 2BC and 2BD, respectively.

Reference is made now to FIG. 2C, presenting the second means to maneuver blade (1), i.e., rotating it laterally in any predetermined manner so that a side-to-side resection is provided. Hence, when knob (6b) is rotated in the direction and measure (2CA), cable (5) accordingly rotates, simultaneously rotating rod (2) and blade (1) in direction and measure (2CB), wherein curves (2CA) and (2CB) may be either equal or different in angular direction, and the rotation may be either clockwise or counter-clockwise.

Reference is made now to FIG. 3A, illustrating the aforementioned simplified working tool (100) without the outer housing envelope (6A); comprising inter alia flexible blade (1), rectangular rigid rod (2), outer flexible tube (4), movement cable or, alternatively, movement cable assembly (5), control knob (6B), and electrical inlet (8) accommodated in electrical inlet roller (9). Roller (9) is connected to cable (5) and allows a continuous electrical contact through inlet (8) when cable 5 rotates laterally. FIG. 3B presents the same, wherein connector (10) connects cable (5) to rod (2) in a rigid manner, thus ensuring their effective coupling and a continuous electrical contact.

It is in the scope of the present invention wherein the rotational movement of blade (1) is alternatively provided by a means of a plurality of interconnected conic wheels (not shown).

Reference is made now to FIG. 4A, illustrating the distal portion of the aforementioned working tool (100), without presenting outer flexible tube (4). Flexible blade (1) is connected to a rectangular rigid rod (2) at its very distal end or at any adjacent location, wherein its proximal end comprises a rectangular hole affixed around rod (2) by means of sliding bore (1A), here with a rectangular shape similar to the size and shape of the rod's cross-section. Thus, the proximal portion of blade (1) is maneuverable along the longitudinal axis of rode (2) to any predetermined measure. Cup (3) is an adaptor located at the distal end of tube (4), designed to restrain rod (2) radially while enabling rod (2) to enter and exit tube (4) in the longitudinal axis of the device. In addition cup (3) is also designed to adapt to and fill the gap between said relatively small, polygonal rods cross-section and the bigger and rounded inner bore of tube (4, not shown). Connector (10) is further illustrated to present the coupling site of cable (5) and rod (2). FIG. 4B shows blade (1) of working tool (100) bending so that a curved blade is obtained; and FIG. 4C shows the same blade rotating laterally such that a side-to-side rotation is provided.

It is acknowledged in this respect that a compressing spring may potentially envelop rectangular rod (2) adapted to either push or pull said proximal loose portion of the flexible blade (1).

It is further acknowledged that blade (1) may be comprised of any suitable structure, selected in a non-limited manner from a blade-like shape as presented in FIG. 4A-C, deflectable wire or deflectable wire-like filament of any suitable diameter and shape, a wire or wire-like member characterized by a unitendon or multitendon infrastructure; or loop-like symmetrical shapes, toothed or sharpened members, polygonal shaped constructions, spoon-like structures, roller-like, spring-like members or scythe-like non-symmetrical blades or any combination thereof. Various embodiments are presented in FIG. 16. This blade is made of, yet not restricted to metals (e.g., stainless steel, tungsten, niobium chromated metal alloys etc), shape memory alloys (e.g., nickel titanium based alloys), composite materials, polymers or any mixture thereof.

According to the present invention, blade (2) is adapted for either cold resection, i.e., to cutting of biological tissues mechanically, or hot resection, i.e., cutting of biological tissues, e.g., by means of a blade, adapted to forward and backwards knife movement by any suitable diathermia or other heated or cooled means known in the art; RF or any other commercially available vibrational means and/or any combination thereof. Hence, said blade may be either sharp or blunt, or a combination thereof.

Reference is made now to FIG. 5, illustrating one embodiment of the present invention wherein movement cable (5) is a spring assembly (50) comprising a clockwise coiled inner spring (50B) and counter-clockwise coiled outer spring (50A) enveloping said second spring of the inner core and vice versa. Said spring assembly (50) is forced to rotate in a certain direction (5A), here clockwise. Thus, the outer spring (50A) is coiled to become thinner and the inner spring (50B) is coiled to become thicker so that the two springs clutch each other firmly to minimize flexibility in the clockwise angular direction along the longitudinal main axis. A third spring may be added to envelope both said springs, coiled according to the inner spring, thus minimizing flexibility in both angular directions clockwise and counter-clockwise.

Reference is made now to FIG. 6A, schematically presenting the working tool (60) according to yet another embodiment of present invention. The distal portion of tool (60) is generally similar to the one defined and described in tool (100) above, and comprises inter alia flexible blade (1), rectangular rigid rod (2), cup member (3), outer flexible tube (4), and movement cable (5) or interchangeably spring assembly (50). The control box (6) of tool (60) comprises inter alia a housing element (6A) adapted to be handled by the user, an electrical inlet (8), a rotating knob (6B) for rotating blade (1) in any desired lateral maneuver, and bending knob (6C) for bending blade (1) so a curved blade is provided.

FIG. 6B presents the same tool (60) wherein bending knob (6C) is twisted backwards along threads (6D), puffing movement cable (5) and rectangular rode (2) backwards, so flexible blade (1) is forced to bend along its longitudinal axis to form a curved blade. FIG. 6C presents the same tool (60) wherein rotating knob (6B) is rotated, forcing blade (1) to maneuver laterally in a predetermined angular course.

Reference is made now to FIG. 7A, schematically presenting the aforementioned working tool (60) without the box housing (6A). Said tool comprises a bending knob thread (6D), a bending knob (6C), lock (12) and a rotating knob (6B). FIG. 7B presents the same apparatus without showing the bending knob thread (6D) and its bending knob (6C).

Reference is made now to FIG. 8A, schematically presenting the working tool (80) according to yet another embodiment of present invention. The distal portion of tool (80) is generally similar to the one defined and described in tools (60) and (100) above, and comprises inter alia flexible blade (1), rectangular rigid rod (2), cup member (3), outer flexible tube (4), and movement cable (5) or interchangeably spring assembly (50); the control box (6) comprises inter alia bending knob (6C) and its bending knob thread (not shown), a handle (13) maneuvered forwards and backwards along the longitudinal axis in a recess (not shown). An optional palm rest (14) is located at the extreme proximal end, adapted to accommodate the thumb or palm of the user. Said rest may be designed to any proper configuration, such as a ring member, a designed niche, a nest-like member etc. Bending knob (11) is twisted or rolled by the user to either pull or push tube (4) thus lengthening or shortening the distance between cap (3) and control box (6), movement cable (5) or spring assembly (50), and being rigidly connected along its longitudinal axis between rod (2) and control box (6) will then either pull or release rod (2), thus adjusting flexible blade (1) to curve to a desired measure.

FIG. 8B shows working tool (80) with a bending maneuver. Bending knob (11) is twisted by the user to either pull or push tube (4) thus lengthening or shortening the distance between cap (3) and control box (6), movement cable (5) or spring assembly (50), and being rigidly connected along its longitudinal axis between rod (2) and control box (6) will then either pull or release rod (2), thus adjusting flexible blade (1) to curve to a desired measure; a tension spring (20) (not shown) is added to apply constant tension to movement cable (5) thus ensuring smooth operation. FIG. 8C illustrates the same, with a rotating maneuver. Handle (13) is maneuvered by the user to the proximal end to rotate cable (5) or spring assembly (50) and thus to enable rotational maneuver of the blade so that a side-to-side resection is provided.

Reference is made now to FIG. 9A, schematically presenting the aforementioned working tool (80) without showing the box housing and the handle. Said tool comprises inter alia bending knob (6C), cable (5), twisted rectangle (18), connector (15), sliding bolt (16), at least one returning spring (17) adapted to return handle (13) toward the distal end of tool (80) after its maneuver toward the proximal end of tool (80), an electrical inlet roller (9) and an electrical inlet (8). Reference is made now to FIG. 9B, schematically presenting the aforementioned working tool (80) without showing as in FIG. 9A bend knob (11) and return spring (17). Optional tube tension spring (20) is located at the distal portion of movement cable (5), adjacent to connector (10). Tube pusher (19) has a tube pushing thread (19A) and a protruding pin member adapted to fit a slot in the tool's housing (6, not shown), pin member (19) is utilized to disable angular rotation of tube pushing thread (19A). The rotating mechanism comprises inter alia the following components: a handle member (13), a sliding bolt (16), and a twisted rectangle (18). By maneuvering the handle (13) along the longitudinal axis of the tool, sliding bolt (16) which is rigidly coupled to handle (13) cannot rotate angularly, and thus is simultaneously pushed or pulled, rotating twisted rectangle. (18). Connector (15) rigidly connects twisted rectangle (18) to the movement cable (5) or spring assembly (50). Tube pusher (19) is maneuvered along said longitudinal axis by means of thread (19A) driven by the knob (11). Tube pusher (19) is rotationally locked to housing (6) by means of slot and pin member assembly.

Reference is made now to FIG. 10A, schematically presenting a lateral cross section of the whole aforementioned working tool (80). FIG. 10B presents the same, wherein the central rotating and bending mechanism, i.e., rectangular rod (2), tube (4), tube pusher thread (19A), sliding bolt (16), twisted rectangular (18) etc, is projected without the enveloping ingredients. It is acknowledged in this respect that this central rotating and bending mechanism is locked in a longitudinal axis and may only rotate in a predetermined measure, wherein back and forwards movements are restricted by the housing compartment (6).

Reference is made now to FIG. 11, schematically presenting said central rotating and bending mechanism (110) connected at its proximal portion to three electrical contact assemblies, wherein the upper mechanism comprises a electrical inlet roller (9) adapted to accommodate electrical inlet (8); central mechanism comprising a coal pressure contact (21) and its contact spring (22); and a lower mechanism comprising a plurality of connecting plates (23).

Reference is made now to FIG. 12, schematically presenting the very distal end of working tool (120) according to yet another embodiment of present invention, comprising two pull/release cables instead of one cable with two functions, rotating and pull/release movements utilized in the previous embodiments, a control box which is not shown for this embodiment. The extreme distal portion of tool (120) is generally similar to the previously defined and described embodiments above, and comprises inter alia flexible blade (1) and a rectangular rigid rod (2). At the proximal portion of blade (1) a blade bend plate (25) is located, grasped (e.g., either pulled or released) by means of cables (26), which are located at the two sides of plate (25), and fuse into one blade bend cable (27) following part (34) (not shown here). Blade bend spring (24) is utilized to bend blade (1) when cables (26) are released, or partly released. Said plate (25) is maneuvered by means of a blade bend cable (26) and spring (24). In addition, at the proximal end of the drawing, the ends of cable (27), which is utilized, to bend and flatten blade (1) is shown, as is cable (28), which is utilized to rotate blade (1) around the longitudinal axis of said tool.

Reference is made now to FIGS. 13A-13B, schematically presenting said cable-based working tool (120) mode of action. Releasing the blade band cable end (27) forwards towards the proximal direction (See FIG. 13A) simultaneously results in release of the blade band cable (26), hence causing spring (24) to elongate, thus forcing blade (1) to bend. Pulling the blade rotate cable end (28) backwards towards the distal direction (See FIG. 13B) similarly results in rotating the blade clockwise, and vice versa; when cable (27) is pulled blade (1) will simultaneously flatten, and when cable (28) is released blade (1) will simultaneously rotate counter clockwise.

Reference is made now to FIGS. 14A-14C, schematically presenting said cable-based working tool (120) inner construction and mode of action. As illustrated in FIG. 14A, the bending mechanism is comprised inter alia of a blade bending cable (26) maneuvered at its proximal end (27) in a direction parallel to the longitudinal axis, releasing, or partially releasing spring (24) and thus pushing plate (25) forwards so that blade (1) is bended or partially bended, as desired by the user. Also presented in FIG. 14A is the rotating mechanism, which is comprised inter alia of blade rotating cable, terminating at the tool's proximal portion (28), blade rotating spring (35) and rotating spring stopper (36). When said cable (28) is pulled backwards in a direction parallel to the longitudinal axis, said cable passes freely through the center of bend cables connector (34) and connects to rectangular rod (32A) which passes through disk (33) which incorporates a rectangular hole (33a), thus rectangular rod (32a) is unable to rotate angularly around the longitudinal axis of said tool. At its distal end rectangular rod (32a) connects to rectangular twisted rod (32) thus activating its linear movement along the longitudinal axis of the tool and disabling its angular movement. Spring (35) is positioned between disk (33) and spring stopper (36) which is rigidly connected to rectangular rod (32a), when cable (28) is pulled rectangular rod (32a) travels toward the proximal end of said tool, thus further loading preloaded spring (35) and vise versa. Nut (31) incorporates a twisted rectangular hole and is unable to move linearly and thus rotates when twisted rectangular rod (32) is actuated. Nut (31) is rigidly connected to connector (30) thus both are rotated angularly by twisted rectangular rod (32). Rectangular rod (2) is rigidly connected to connector (30) and thus rotates accordingly.

It is further acknowledged that a simple control box may be designed to control the pull release of cables (27) and (28).

A closer view of aforesaid mechanism of bending and rotating is provided in FIGS. 15A-15C. FIG. 15A illustrates the distal portion of tool (120) comprising blade (1) on rod (2), wherein blade bend plate (25) is clasped against the distal portion of the blade by means of blade bend spring (24). The proximal end of blade (1) slides along rode (2) by means of quadrangle bore (1A). Cup tube (29) forces said spring to its illustrated location. By pulling or releasing blade bend cable (26), plate (25) is shifted backwards or forwards, to erect or deflect the blade so that the desired curved shape is obtained. FIG. 15B illustrates the central portion of tool (120) comprising cup tube (29), nut connector (30), twisted rectangular rod (32) distal portion and the rectangular rod (32a) extruding from it, nut (31), and blade rotate spring (35). FIG. 15C shows the extreme distal portion of the same, underlining the position of the bend cable connector (34).

Lastly, reference is made to FIGS. 16A-16E, presenting in a non-limiting manner various non-wire blades and wire-like blades, namely non-symmetrical saw-like blade, (16A), pointed-edge blade (16B), symmetrical polygonal blade (16C), deflectable filament-type blade (16D) and rotating multiply-filamented blade, here double-wired structure adapted to bend and rotate (16E).

The present invention also provides a novel method for lateral resection of biological tissues by means of a working tool with flexible blade of an adjustable curve size. The core of the aforesaid method consists of the sequential steps of inserting the distal end of said tool into a body cavity, and positioning the maneuverable blade in contact with or adjacent to the tissue to be resected; bending said blade to a predetermined measure; rotating said blade in a lateral movement such that a side-to-side resection is obtained; and then, retracting said blade and withdrawing the tool outside said body cavity.

It is according to one embodiment of the present invention wherein the aforementioned method of lateral resection is based on a working tool with a flexible blade of an adjustable curve size. This tool is elongated and narrow and has a distal end, which is inserted into a body cavity, and a proximal end, which is adjacent to a user; the longitudinal axis of said maneuverable blade is parallel to the longitudinal axis of the said tool. It is further characterized by both a bending mechanism, adapted to bend said blade to a predetermined measure, and a rotating mechanism, adapted to rotate said blade in a lateral movement such that a side-to-side resection is obtained. Such a useful method is provided by the working tools as defined and described in any of the above embodiments of the present invention and in their descriptive figures.

It is according to yet another embodiment of the present invention wherein the aforementioned method of lateral resection comprises the steps of inserting the distal end of said tool into a body cavity, and positioning the maneuverable blade in contact with or adjacent to the tissue to be resected; bending said blade to a predetermined measure; supplying high frequency electrical current to said blade while rotating said blade in a lateral movement such that a side-to-side resection is obtained; switching off the current when the incision procedure is completed; and then retracting said blade and withdrawing the tool outside said body cavity. Such a method is especially useful for facilitated (hot) resections as defined above.

This facilitated (hot) method is especially useful when the working tool is adapted for lateral resection of biological tissues by providing a flexible blade with an adjustable curve size; said tool is elongated and narrow and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained. Such a working tool may be selected from any embodiment defined and described in the present invention and in its appended figures.

We claim:

1. A flexible rod-like working tool for lateral resection of biological tissues in or adjacent to narrow body cavities and lumens by means of a flexible blade with adjustable curve size; said rod-like tool having a rigid extreme distal end, adapted to be inserted into either a linear or curved body cavity, and a proximal end, located adjacent to a user; said flexible blade having a distal end rigidly immobilized to a rod and whose proximal end comprises a hole affixed around said rod by means of a sliding bore with a polygonal profile such that said blade can slide along a longitudinal axis solely to a predetermined point; the longitudinal axis of said blade being parallel to a longitudinal axis of the said tool such that the movement of said blade is restricted only in a linear movement along said longitudinal axis of said rod, disabling its angular movement around the longitudinal axis of said rod; said working tool comprising:
a. a bending mechanism, adapted to adjust the bending of said blade to a predetermined measure;
b. a rotating mechanism, adapted to rotate said blade in a lateral bi-directional movement in respect to the resected tissue such that a side-to-side resection is obtained; and
c. a plurality of maneuvering means located at its proximal portion especially adapted to maneuver said flexible blade by bending it and/or rotating it laterally.

2. The working tool according to claim 1, wherein the shape of the blade is selected from blade, wire-like, snare or loop-like symmetrical, asymmetrical shapes, toothed or sharpened members, polygonal shaped constructions, roller-like, spoon-like structures, spring-like members or scythe-like non-symmetrical blades or any combination thereof.

3. The working tool according to claim 1 useful for cold resection; wherein the maneuverable blade is a sharp razor-like member adapted to cut biological tissues mechanically.

4. The working tool according to claim 1 useful for cold resection; wherein the maneuverable blade is adapted to forward and backwards knife movement, vibrational motion, or a combination thereof.

5. The working tool according to claim 2 wherein the blade is made of metals selected from stainless steel, tungsten, niobium, chromatic alloys or shape memory alloys; polymers; composite materials or any mixture or combination thereof.

6. The working tool according to claim 1 useful for facilitated (hot) resection;
additionally comprising means for diathermia; cryo-therapy; RF or any other vibrational means; or any combination thereof.

7. The working tool according to claim 1 wherein said maneuvering means are selected from at least one control knob, handle, endless cable or any combination thereof.

8. The working tool according to claim 1 additionally comprising at least one handle located at its proximal portion adapted to maneuver the flexible blade located at the distal end by both bending it and rotating it laterally.

9. A disposable working tool according to claim 1.

10. The working tool for endoscopic resection according to claim 1, comprising:
a. a rigid rod located at the extreme distal portion of said tool;
b. a flexible blade;
c. a control box comprising inter alia a housing element, and at least one control knob;
d. an endless movement cable connecting rod at the distal portion of the tool with the control knob located at its proximal portion; and
e. an outer flexible tube with a circular bore encompassing the proximal portion of said rod and the said movement cable; wherein by either pushing or pulling the said control knob along the longitudinal main axis of the tool, said movement cable and said rod are simultaneously pulled or pushed, curving or flattening the said flexible curve, and further wherein by twisting said control knob around said longitudinal main axis, said movement cable and said rod are simultaneously rotated, rotating the said flexible curve along a predetermined course.

11. The working tool according to claim 10 additionally comprising an electrical inlet adapted to provide the blade with effective diathermia, cryo-therapy or RF means; wherein a free and continuous electrical communication is provided by the said electrical inlet, to the movement cable, rectangular rod and flexible blade.

12. The working tool according to claim 11, wherein the movement cable of the electrical inlet is rolled over an electrical inlet cable allowing the movement cable to rotate freely.

13. The working tool according to claim 11, wherein the electrical inlet is in communication with a coil pressure contact and its contact spring.

14. The working tool according to claim 11, wherein the electrical inlet is in communication with a plurality of connecting plates.

15. The working tool according to claim 10, wherein at least a portion of the movement cable is a spring assembly comprising a clockwise coiled inner spring and counter-clockwise coiled outer spring enveloping said second spring of the inner core and vice versa.

16. The working tool as defined in claim 10 wherein the control box comprises inter alia a rotating knob for rotating the blade in any predetermined lateral maneuver, and a bending knob twistable on a bending knob thread for bending it to obtain a predetermined curve; and further wherein said bending knob is twisted backwards, pulling the movement cable or spring assembly and rod backwards, so that the flexible blade is forced to bend along its longitudinal axis to form a curved blade.

17. The working tool as defined in claim 10 wherein the control box comprises
a. a bending knob twistable on a bending knob thread for bending it to obtain a predetermined curve; and further wherein said bending knob is twisted backwards, pulling movement cable or spring assembly and rod backwards, so that flexible blade is forced to bend along its longitudinal axis to form a curved blade;
b. a handle assembly comprising
i. a handle maneuvered forwards and backwards along the longitudinal axis in a recess at the housing;
ii. a tube pusher with a tube pushing thread and a protruding pin member adapted to fit a slot in the tool's housing;
iii. a twisted rectangle;
iv. a cable and screw connector rigidly connecting said twisted rectangular to the movement cable or spring assembly;
v. a sliding bolt;
vi. at least one return spring adapted to either pull or push said handle after its maneuver; and
vii. a palm rest located at the extreme proximal end, adapted to accommodate the thumb or palm of the user;
when the handle is maneuvered by the user along the longitudinal axis of the tool, it simultaneously pushes or pulls the sliding bolt, thus rotating twisted rectangle and transforms a linear movement into a rotational maneuver of the blade so that a side-to-side resection is provided.

18. The working tool as defined in claim 10 wherein a blade band plate is located at the distal portion of the rod; said plate is grasped either by pulling or pushing by means of a blade bend spring; said plate is maneuvered by means of at least one blade bend cable with at least two proximal ends; and further comprising a blade bending cable end and a blade rotating cable end; wherein said blade bending cable is maneuvered at its proximal end in a direction parallel to the longitudinal axis, releasing said spring and thus pushing plate forwards so that blade is bended in a predetermined manner; and further wherein the blade rotating cable, terminating at said tool's proximal portion is pushed backwards in a direction parallel to the longitudinal axis, said cable, via bend cables connector and a rectangular rod connected to it, rotates a twisted rectangular rod by means of a twisted rectangular, nut and nut connector rotating assembly, such that said nut connector is in communication at its distal portion with a cap tube so that said cable longitudinal movement is translated to said blade in a predetermined lateral rotation.

19. A method for lateral resection of biological tissues by means of a working tool with a flexible blade of an adjustable curve size, operated by:
a. inserting the distal end of said tool into a body cavity, and positioning the maneuverable blade in contact with or adjacent to the tissue to be resected;
b. bending said blade to a predetermined measure;
c. rotating said blade in a lateral movement such that a side-to-side resection is obtained; and,
d. retracting said blade and withdrawing the tool outside said body cavity.

20. The method according to claim 19, wherein the working tool is adapted for lateral resection of biological tissues by means of a flexible blade of an adjustable curve size; said tool is elongated and narrow and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained.

21. A method for lateral resection of biological tissues by means of a working tool with a flexible blade of an adjustable curve size comprising the steps of inserting the distal end of said tool into a body cavity, and positioning the maneuverable blade in contact with or adjacent to the tissue to be resected, bending said blade to a predetermined measure; rotating said blade in a lateral movement such that a side-to-side resection, rotation or other lateral maneuver is obtained; and retracting said blade and withdrawing the tool outside said body cavity, wherein said working tool is defined in claim 1 or in any of its dependent claims.

22. The method according to claim 19, comprising the steps of: inserting the distal end of said tool into a body cavity, and positioning the maneuverable blade in contact with or adjacent to the tissue to be resected; bending said blade to a predetermined measure; g. supplying high frequency electrical current to said blade while rotating said blade in a lateral movement such that a side-to-side resection is obtained; switching off the current when the incision procedure is completed; and, retracting said blade and withdrawing the tool outside said body cavity.

23. The method for lateral resection of biological tissues according to claim 21, wherein the working tool is adapted for lateral resection of biological tissues by means of a flexible blade with an adjustable curve size; said tool is elongated and narrow and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained.

24. The method according to claim 21 comprising the steps of inserting the distal end of said tool into a body cavity, and positioning the maneuverable blade in contact with or adjacent to the tissue to be resected; bending said blade to a predetermined measure; supplying high frequency electrical current to said blade while rotating said blade in a lateral movement such that a side-to-side resection is obtained; switching off the current when the incision procedure is completed; and retracting said blade and withdrawing the tool outside said body cavity; wherein said working tool is defined in claim 1.

25. The working tool according to claim 1, especially for lateral resection of urological tissues wherein said rod-like tool has a distal end, adapted to be inserted into the urethra.

26. The working tool according to claim 25, especially adapted to be inserted into the body throughout a hollow facilitating means, especially laparascopes or trocars, so as said blade is introduced to urological organs and/or male or female reproduction organs, especially the kidney, urethral bladder, prostate or the urinal tracks.

27. The working tool according to claim 1, especially for lateral resection of male or female reproduction organs wherein said rod-like tool has a distal end, adapted to be inserted into the urethra.

28. A non-urological working tool according to claim 1 for lateral resection of biological tissues by means of a flexible blade with adjustable curve size; said rod-like tool has a distal end, adapted to be inserted into any body cavity, excluding the urological system, yet including gastrointernal system, respiratory system, and large blood vessels.

29. The working tool according to claim 28, especially adapted to be inserted into the body throughout a hollow facilitating means, especially laparascopes, or trocars, so as said blade is introduced to non-urological organs, especially to the gastrointernal system, respiratory system, and large blood vessels.

30. The working tool according to claim 1, especially for lateral resection of respiratory system wherein said rod-like tool has a distal end, adapted to be inserted into the respiratory system.

31. A resectoscope comprising the working tool for urological resection as defined in claim 1.

32. The method according to claim 19, wherein the step of inserting comprises applying the distal end of said tool into a hollow facilitating means being selected from a group including laparascopes, trocars or any other hollow surgical means adapted to introduce said distal end to urological organs, especially the kidney, urethral bladder, prostate or the urethra tracks.

33. The method according to claim 19, by means comprising applying a working tool adapted for lateral resection of urological tissues by means of a flexible blade of an adjustable curve size; said tool is elongated and narrow and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained.

34. A method for lateral resection of urological tissues by means of a working tool with a flexible blade of an adjustable curve size comprising the steps of inserting the distal end of said tool into a body cavity, and positioning the maneuverable blade in contact with or adjacent to the tissue to be resected, bending said blade to a predetermined measure; rotating said blade in a lateral movement such that a side-to-side resection, rotation or other lateral maneuver is obtained; and retracting said blade and withdrawing the tool outside said urological cavity, wherein said working tool is defined in claim 1.

35. The method according to claim 34, wherein the step of inserting comprises applying the distal end of said tool into a hollow facilitating means being selected from a group including laparascopes, trocars or any other hollow surgical means.

36. The method according to claim 19, comprising the steps of:
a. inserting the distal end of said tool into an urological cavity or into hollow facilitating means, and positioning the maneuverable blade in contact with or adjacent to the tissue to be resected;
b. bending said blade to a predetermined measure;
c. supplying high frequency electrical current to said blade while rotating said blade in a lateral movement such that a side-to-side resection is obtained;
d. switching off the current when the incision procedure is completed; and,
e. retracting said blade and withdrawing the tool outside said urological cavity.

37. The method for lateral resection of biological tissues according to claim 36, additionally comprising applying a working tool which is adapted for lateral resection of urological tissues by means of a flexible blade with an adjustable curve size; said tool is elongated and narrow so as it is insertable into the urethra or to a hollow surgical facilitating means, and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained.

38. The method according to claim 37, comprising the steps of inserting the distal end of said tool into the uretra, and positioning the maneuverable blade in contact with or adjacent to the tissue to be resected; bending said blade to a predetermined measure; supplying high frequency electrical current to said blade while rotating said blade in a lateral movement such that a side-to-side resection is obtained; switching off the current when the incision procedure is completed; and retracting said blade and withdrawing the tool outside said urological cavity; wherein said working tool is defined in claim 1 or in any of its dependent claims.

39. The method according to claim 38, wherein the step of inserting comprises applying the distal end of said tool into a hollow facilitating means being selected from a group including laparascopes, trocars or any other hollow surgical means adapted to introduce said distal end to urological organs, especially the kidney, urethral bladder, prostate or the urethra tracks.

40. A method for lateral resection of biological tissues, excluding urological tissues yet including gastrointernal system, respiratory system, and large blood vessels, by means of a working-tool with a flexible blade of an adjustable curve size, operated by:
a. inserting the distal end of said tool into a body cavity excluding the urological cavity yet including gastrointernal cavity, respiratory cavity, and large blood cavity;
b. positioning the maneuverable blade in contact with or adjacent to the tissue to be resected;
c. bending said blade to a predetermined measure;
d. rotating said blade in a lateral movement such that a side-to-side resection is obtained; and,
e. retracting said blade and withdrawing the tool outside said body cavity.

41. The method according to claim 19, by means wherein the step of inserting is introducing a hollow facilitating into a body cavity, excluding the urological cavity yet including gastrointernal cavity, respiratory cavity, and large blood cavity, said means is means being selected from a group including laparascopes, trockars or any other hollow surgical means, adapted to introduce said distal end to the body cavity, excluding the urological cavity, yet including gastrointernal cavity, respiratory cavity, and large blood vessels cavity.

42. The method according to claim 19, by means comprising a working tool adapted for lateral resection of biological tissues by means of a flexible blade of an adjustable curve size; said tool being elongated and narrow and having a distal end, which is inserted into a body cavity, and a proximal end, which is adjacent to a user; the longitudinal axis of said maneuverable blade being parallel to the longitudinal axis of said tool; wherein said working tool is characterized by both a bending mechanism, adapted to bend said blade to a predetermined measure; and rotating a mechanism, adapted to rotate said blade in a lateral movement such that a side-to-side resection is obtained.

43. A method for lateral resection of biological tissues excluding the urological tissues yet including gastrointernal system, respiratory system, and large blood vessels by means of a working tool with a flexible blade of an adjustable curve size comprising the steps of inserting the distal end of said tool into a body cavity, and positioning the maneuverable blade in contact with or adjacent to the tissue to be resected, bending said blade to a predetermined measure; rotating said blade in a lateral movement such that a side-to-side resection, rotation or other lateral maneuver is obtained; and retracting said blade and withdrawing the tool outside said body cavity, wherein said working tool is defined in claim 1.

44. The method according to claim 43, comprising the steps of:
a. inserting the distal end of said tool into a body cavity excluding the urological cavity yet including gastrointernal cavity, respiratory cavity, and large blood vessels cavity, and positioning the maneuverable blade in contact with or adjacent to the tissue to be resected;
b. bending said blade to a predetermined measure;
c. supplying high frequency electrical current to said blade while rotating said blade in a lateral movement such that a side-to-side resection is obtained;
d. switching off the current when the incision procedure is completed; and,
e. retracting said blade and withdrawing the tool outside said body cavity.

45. The method for lateral resection of biological tissues according to claim 44, wherein the working tool is adapted for lateral resection of biological tissues by means of a flexible blade with an adjustable curve size; said tool is elongated and narrow and has a distal end, which is inserted into a body cavity, and a proximal end, which is adjacent to a user; the longitudinal axis of said maneuverable blade is parallel to the longitudinal axis of the said tool; wherein said working tool is characterized by both bending mechanism, adapted to bend said blade to a predetermined measure; and rotating mechanism, adapted to rotate said blade in a lateral movement such that a side-to-side resection is obtained.

46. The method according to claim 44, comprising the steps of inserting the distal end of said tool into a body cavity excluding the urological cavity yet including gastrointernal cavity, respiratory cavity, and large blood vessels cavity, and positioning the maneuverable blade in contact with or adjacent to the tissue to be resected; bending said blade to a predetermined measure; supplying high frequency electrical current to said blade while rotating said blade in a lateral movement such that a side-to-side resection is obtained; switching off the current when the incision procedure is completed; and retracting said blade and withdrawing the tool outside said body cavity; wherein said working tool is defined in claim 1.
